(19) United States
(12) Patent Application Publication
(10) Pub. No.: US 2021/0154326 A1
(43) Pub. Date: May 27, 2021

(54) CRISPR-Based Synthetic Gene Circuits as Next Generation Gene Therapy of Inner Ear

(71) Applicant: Arizona Board of Regents on behalf of Arizona State University, Scottsdale, AZ (US)

(72) Inventors: Samira Kiani, Scottsdale, AZ (US); Swechchha Pradhan, Tempe, AZ (US); Farzaneh Moghadam, Tempe, AZ (US); Mo Reza Ebrahimkhani, Scottsdale, AZ (US)

(21) Appl. No.: 16/626,013

(22) PCT Filed: Jun. 26, 2018
(86) PCT No.: PCT/US2018/039580

§ 371 Date: Dec. 23, 2019

Related U.S. Application Data
(60) Provisional application No. 62/524,936, filed on Jun. 26, 2017.
(60) Provisional application No. 62/552,312, filed on Aug. 30, 2017.

(51) Int. Cl. A61K 48/00; C12N 15/113; C12N 15/86; C12N 9/22

(52) U.S. Cl. CPC A61K 48/005; C12N 15/113; C12N 15/86; C12N 9/22; C12N 2830/008; C12N 2330/51; C12N 2710/16643; C12N 2750/14143; C12N 2310/20

(57) ABSTRACT

Aspects of the disclosure relate to synthetic regulatory systems comprising a multifunctional Cas nuclease and at least two guide RNAs (gRNAs) including a truncated gRNA and an multilayered regulatory control element. The synthetic regulatory system modulates endogenous gene expression, including transcriptional repression and transcriptional activation of one or more endogenous genes of a mammalian inner ear cell with multiple safety switches. Also provided herein are methods for modulating hearing sensitivity in damaged cells of the organ of corti.

### CROSS-REFERENCE TO RELATED APPLICATIONS

This application claims the benefit of U.S. Provisional Application Nos. 62/524,936, filed Jun. 26, 2017, and 62/552,312, filed Aug. 30, 2017, each of which is hereby incorporated by reference in its entirety for all purposes.

### STATEMENT REGARDING FEDERALLY SPONSORED RESEARCH OR DEVELOPMENT

This invention was made with government support under D16AP00047 awarded by the Department of Defense. The government has certain rights in the invention.

### BACKGROUND

Warzone noise-induced damage to the cochlea can cause permanent hearing loss, which can be alleviated by stimulating inner ear hair cells to regenerate. In the cochlea, there are several cell types that function cooperatively. Using conventional gene therapy to target hair cells specifically can be a challenge.

Gene therapy offers a permanent and sustainable cure for diseases and injuries affecting warfighters. While genomic research has identified a number of genetic therapy targets that can modify the course of disease, there has been limited translation of genetic therapies into clinical use. For example, current gene therapy techniques face challenges to translation including the potential to target incorrect cells, silencing genes over time, difficulty delivering large genes, high manufacturing cost, and the risk of permanently altering a patient's germline DNA. Clustered Regularly Interspaced Short Palindromic Repeat (CRISPR) systems have recently revolutionized the field of genome editing. Streptococcus pyogenes Cas9 protein can be targeted to any DNA sequence of interest by means of a small guide RNA (gRNA) that can be engineered to carry complementary sequences to target DNA. Once at the target, Cas9 protein can either cleave or bind DNA, depending on whether it is catalytically active or null. CRISPR is paving the way to therapeutic and investigational gene editing and modulation in a variety of organisms, including animals and humans. Most CRISPR-based studies have focused on modulating gRNA expression from ubiquitously active promoters. Efforts to generate additional internal regulatory control over CRISPR, such as limitation of cell type-specific CRISPRs, have been limited. Accordingly, there remains a need in the art for safer, controllable CRISPR-based genetic circuits for safer, controllable therapeutic applications for hearing loss in vivo.

### SUMMARY

Provided herein, in some embodiments, are synthetic genetic circuits and methods of using the same as a platform for safer, controllable gene therapies of inner ear for hearing loss. Such synthetic genetic circuits and methods can broadly be applied to many injuries sustained by US warfighters and the general populace. These architectures and methods are based, in part, on Clustered Regularly Interspaced Palindromic Repeats (CRISPR) systems. The CRISPR regulatory devices of the present disclosure are controllable in cells (e.g., human cells).

In a first aspect, provided herein is a synthetic regulatory system comprising (a) a nucleotide sequence encoding a Cas nuclease; (b) at least two guide RNAs (gRNAs) comprising a first gRNA of 15 or less nucleotides (nt) in length and a second gRNA of 16 or greater nt in length, wherein the first gRNA is complementary to at least a portion of an endogenous gene, wherein the first gRNA is operably linked to a CRISPR-responsive promoter; and (c) a multilayered regulatory control element comprising (i) a first ligand-responsive ribozyme comprising a sensor component capable of detecting the presence or absence of a cell type-specific or small molecule signal and an actuator component; and (ii) a second layer comprising a ligand-responsive nuclease or regulatory polypeptide capable of cleaving and disabling the synthetic regulatory system in the presence of a second ligand; wherein the nucleotide sequence encoding the Cas nuclease, the at least two gRNAs, and the multilayered regulatory control element comprise a single amplicon.

In some embodiments, the second layer comprises a TALE nuclease or a zinc finger nuclease (ZFN) fused to a miRNA 183-responsive actuator, whereby, in the presence of miRNA 183, expression of the TALE nuclease or ZFN is inhibited and the amplicon remains intact. In some embodiments, the second layer comprises a TALE or Tet repressor fused to a small molecule-responsive degradation tag, whereby, in the presence of the small molecule, degradation of the TALE or Tet repressor promotes expression of the first gRNA and cleavage of the amplicon. In some embodiments, the small molecule-responsive degradation tag is a SMASH tag or Degron-Shield1 system.

The Cas nuclease can be Cas9. The endogenous gene can be selected from the group consisting of Atoh, BDNF, Hes1, Hes5, and HGF. The amplicon can further comprise a nucleotide sequence encoding a MS2 bacteriophage coat protein and the first gRNA can comprise a MS2 target sequence. The MS2 bacteriophage coat protein can be fused to transcriptional activation domain VPR-P65-HSF1.

In some embodiments, one or more of the at least two gRNAs are operably linked to a U6 promoter. In some embodiments, the Cas nuclease is fused to a functional domain selected from the group consisting of a transcriptional activator, a transcriptional repressor, methyltransferase and a nuclease cleavage domain. In some embodiments, the Cas nuclease is Cas9 fused to a functional domain, wherein the nucleic acid sequence encoding Cas9 is split into two halves and fused to a FKBP/FRB domain In some embodiments, the Cas nuclease is an allosteric Cas9, wherein the presence of ramapycin or tamoxifen mediates assembly of functional Cas9 nuclease to enable temporal control over initiation of CRISPR function in vivo. In some embodiments, the functional domain comprises one or more transcriptional activators selected from the group consisting of VPR, VP64, P65, and HSF1. In some embodiments, the functional domain comprises one or more transcriptional repressors selected from the group consisting of Kruppel associated box (KRAB) and KRAB-MeCP2.

In some embodiments, the synthetic regulatory system further comprises a delivery vector. In some embodiments, the delivery vector is an exosome. In some embodiments, the exosome comprises a cell-specific or tissue-specific ligand or receptor. In some embodiments, the delivery vector is a viral delivery vector selected from the group consisting of Herpes Simplex virus, retrovirus, lentivirus, adenovirus, adeno-associated virus, and baculovirus DNA. In some embodiments, the viral delivery vector is Herpes Simplex Virus 1 (HSV1).

In some embodiments, the Cas nuclease is a S. aureus Cas9 nuclease or a S. pyogenes Cas9 nuclease.

In a second aspect, provided herein is a method of modulating endogenous gene expression in an inner ear cell, the method comprising introducing into an inner ear cell the synthetic regulatory system described herein, wherein the single amplicon is provided in a delivery vector. In some embodiments, the inner ear cell is an inner hair cell, an outer hair cell, or a inner ear supporting cell. In some embodiments, modulating comprises one or more of gene activation, gene repression, and gene inactivation. In some embodiments, the delivery vector is an exosome. In some embodiments, the exosome comprises a cell-specific or tissue-specific ligand or receptor. In some embodiments, the delivery vector is a viral delivery vector selected from the group consisting of Herpes Simplex virus, retrovirus, lentivirus, adenovirus, and adeno-associated virus. In some embodiments, the viral delivery vector is Herpes Simplex Virus 1 (HSV1). In some embodiments, introducing the synthetic regulatory system comprises transfection or electroporation.

In a third aspect, provided herein is a polynucleotide sequence comprising (a) a nucleotide sequence encoding a Cas nuclease; (b) at least two guide RNAs (gRNAs) comprising a first gRNA of 15 of less nucleotides (nt) in length and a second gRNA of 16 or greater nt in length, wherein the first gRNA is complementary to at least a portion of an endogenous gene, wherein the first gRNA is operably linked to a CRISPR-responsive promoter; and (c) a multilayered regulatory control element comprising (i) a first ligand-responsive ribozyme comprising a sensor component capable of detecting the presence or absence of a cell type-specific or small molecule signal and an actuator component; and (ii) a second layer comprising a ligand-responsive nuclease or regulatory polypeptide capable of cleaving and disabling the synthetic regulatory system in the presence of a second ligand; wherein the nucleotide sequence encoding the Cas nuclease, the at least two gRNAs, and the multilayered regulatory control element comprise a single amplicon. In some embodiments, the polynucleotide is part of a vector. In some embodiments, the vector is contained within a host cell.

### DESCRIPTION OF THE DRAWINGS

FIG. 1 is a table presenting therapeutic advantages of the synthetic genetic circuits and methods described herein over previous gene therapy approaches for hearing loss.

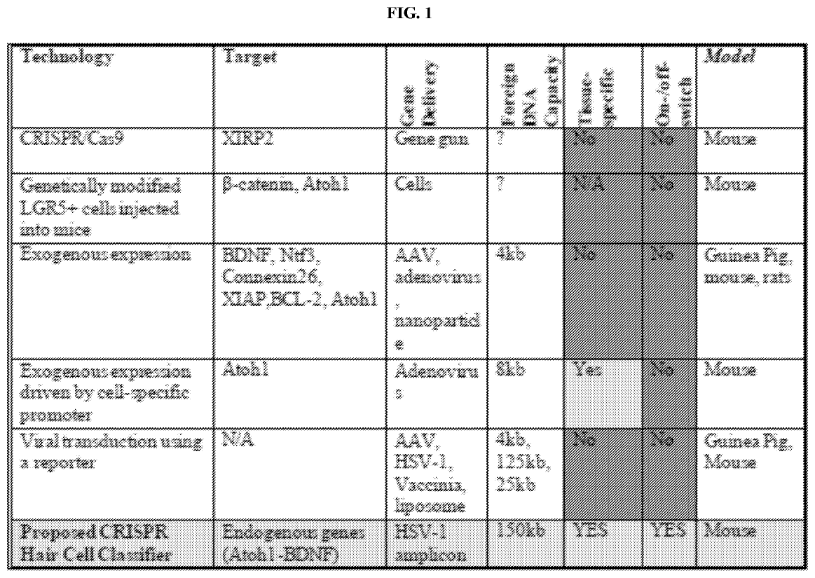

FIG. 2 illustrates an exemplary method for testing gRNAs for gene activation and repression of endogenous genes in vivo. Inner ear-specific genetic circuits are injected into mice via viral vesicles. Hearing loss is induced in the mice using antibiotics. In some cases, the inner ear-specific genetic circuits comprise Cas9, MS2-VPR, and 14 nt gRNAs having MS2 target sites, where expression is driven by Pouf4f3 or Prestin promoters. Multiple gRNAs can be expressed from U6 promoters after further processing by a Csy4 endoribonuclease (from Myosin VIIa promoter). In some cases, a TALE nuclease is expressed in cells that have low miRNA 183 cluster (outside the inner ear) and destroys amplicons by cleaving the amplicon. Another TALE repressor (TALER) that is fused to a degradation/destabilization tag (SMASh) inhibits the expression of 20-nt gRNA. Upon addition of small molecule inducers, this repressor is degraded leading to the expression of 20-nt gRNA and Cas9 nuclease-mediated destruction of the amplicon.

FIG. 3 demonstrates isolation and staining of organ or corti from adult mouse.

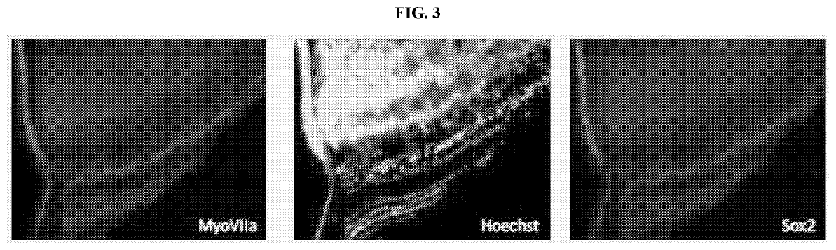

FIG. 4 is a series of images demonstrating the distribution of MyoVIIa in an organ of corti explant from C57BL6 mice (P5). Samples were imaged under confocal microscopy using immersion oil lenses: 20×, 40×, 63×, and 100×.

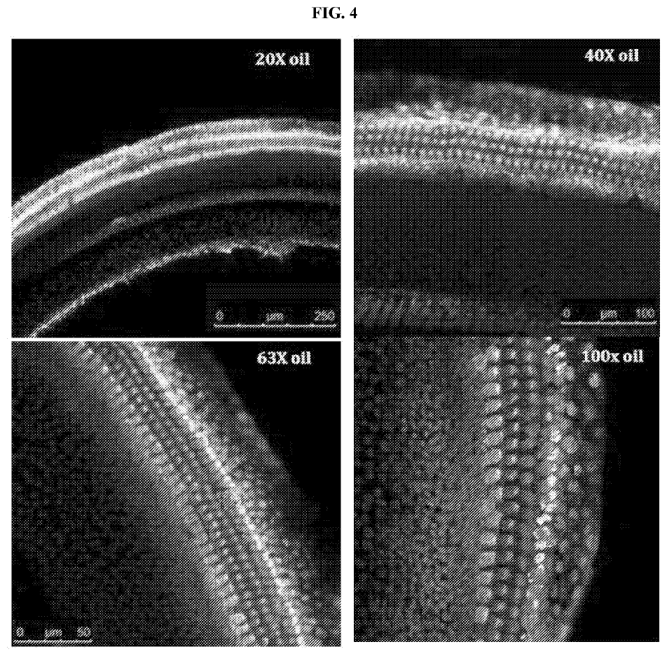

FIGS. 5A-5C demonstrate in vitro activation of ATOH1 and BDNF gene expression using a synthetic genetic circuit described herein. (a) qRT-PCR analysis of ATOH1 and (b) BNDF RNA level relevative to untransfected controls. Raw cells were transfected with one, two or four gRNAs against ATOH1 or BDNF or three gRNAs against these genes, together with the Cas9 activation complex. After 72 hours, cells were lysed for RNA extraction and qRT-PCR analysis. (c) Experiment with triple gRNA combination. A and B refer to gRNAs targeted against ATOH1 or BDNF loci, respectively.

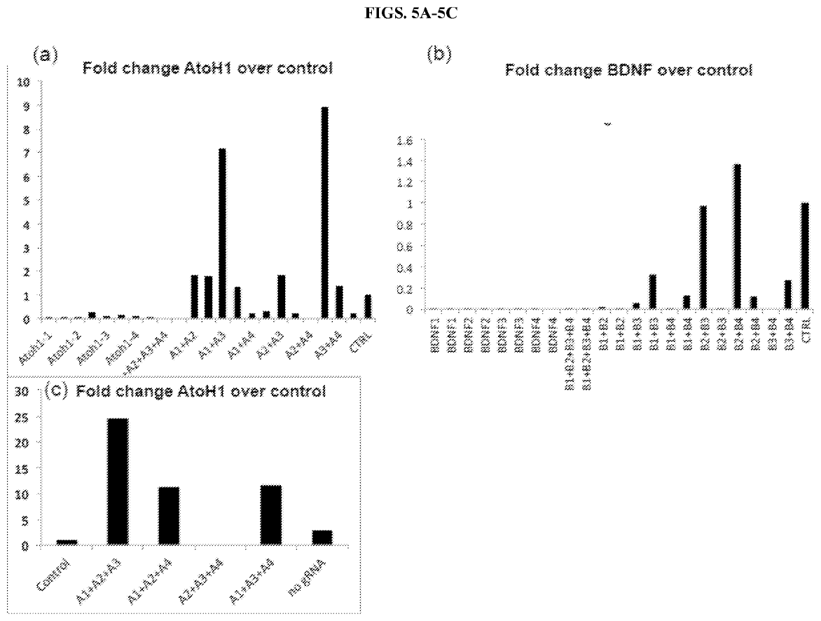

FIGS. 6A-6C are schematic illustrations of a TALER-SMASh Kill Switch. (a) Expression of TALER-SMASh represses the expression of 20-nt gRNA that, in the presence of Cas9, cuts and destroys the amplicon. (b) In this experiment, we tested the function of TALER using an EYFP protein under the control of a TALER repressor promoter. (c) As expected, the presence and absence of TALER can turn on and off the expression of EYFP, respectively.

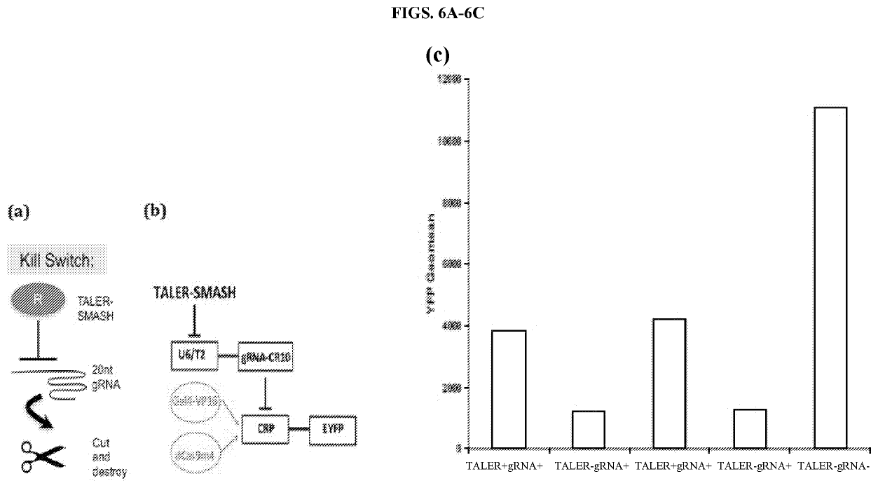

FIGS. 7A-7B demonstrate titration of Exo-AAV1-GFP to determine viral genome copy numbers using q-PCR for GFP target region. a) Standard curve generated using different known concentrations of GFP plasmids which was used to titer concentration of Exo-AAV1-GFP. b) Table showing concentration of Exo-AAV1-GFP which was calculated on the basis of standard curve generated and the correlating genome copy numbers of the virus per mL.

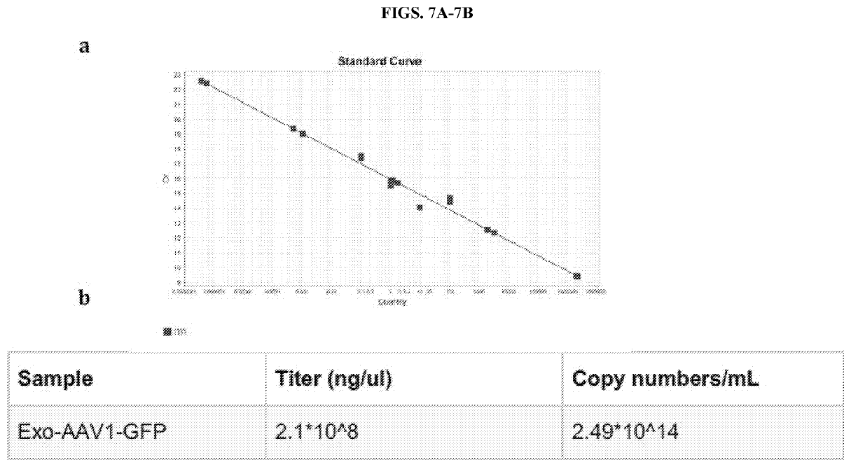

FIGS. 8A-8E demonstrate organ of corti explants from C57BL6 mice (P3) infected with HSV1-EGFP. The virus was added to the cultures on second day at different amounts as shown in the picture: a) Negative control: 0 Units b) 10\hat{ }7 units c) 10\hat{ }6 units d) 100 units e) 10 units. The explants were incubated for 72 hours before staining them with MyoVIIa for hair cells, Sox2 for supporting cells and GFP for HSV1 infected cells. The explants were counterstained with Hoechst which labels all nuclei. Images were taken using epifluorescent microscope.

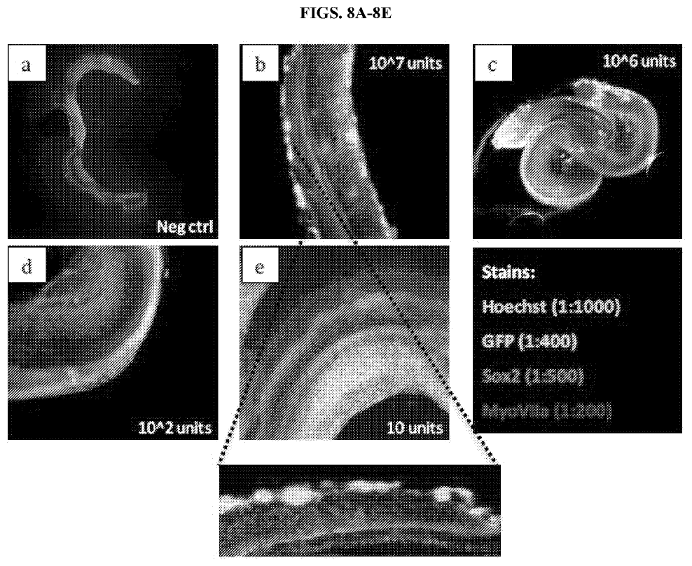

FIG. 9 demonstrates an organ of corti explant from C57BL6 mice (P3) infected with Exo-AAV1-GFP-BFP. 30 μl of undiluted viral suspension was added to the culture on second day followed by 72 hours incubation prior to staining. Images were taken with confocal microscope using from left to right 20×, 40×, and 60× lenses. The images show distribution of MyoVIIa which stains for hair cells, Sox2 for supporting cells and GFP for Exo-AAV1 infected cells. The cells were counterstained with Hoechst which labels all nuclei.

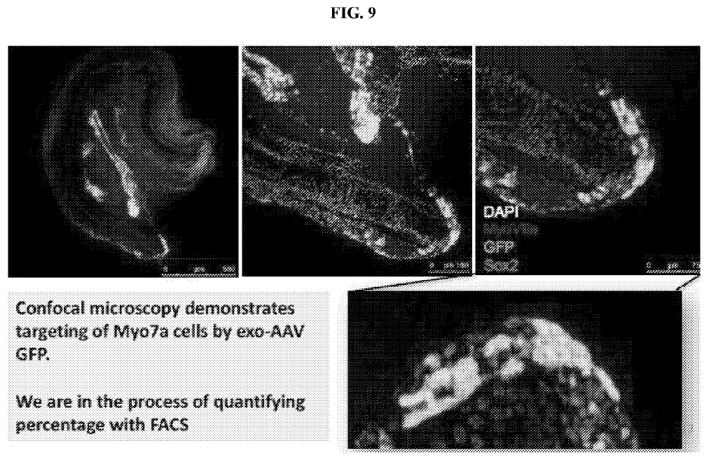

FIGS. 10A-10C demonstrate organ of corti explants from C57BL6 mice (P3) treated with kanamycin. a) Negative control: Organ of corti after three days in kanamycin free culture medium. TUNEL assay with Alexa Fluor® 594 dye (red in the figure above) was utilized to detect the fragmented DNA. After the TUNEL reaction, the cells were counterstained with Hoechst 33342 (gray). Hair cells (indicated by arrows) were negative for Alexa Fluor 594 dye and therefore no apoptotic cells were detected. b) Kan1 Treatment (1 mM Kanamycin): Organ of corti showing apoptotic hair cells after kanamycin treatment. The images were taken 2 days after kanamycin (1 mM) was added to the culture medium. Hair cells (indicated by arrows) were positive for Alexa Fluor 594 dye indicating apoptosis.

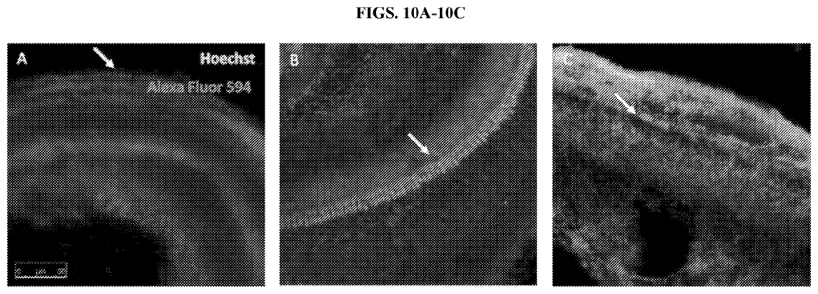

FIG. 11 demonstrates organ of corti explants from C57BL6 mice (P3) treated with 1 mM or 5 mM kanamycin and subjected to a TUNEL assays with Alexa Fluor® 594 dye.

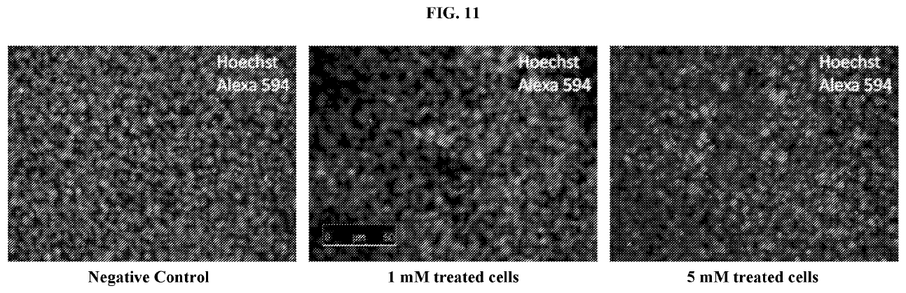

FIG. 12 illustrates a TALER-SMASh Kill Switch and YFP expression.

FIG. 13 is a graph providing a side by side comparison of different CRISPR activators in HEK293 cells. Cells were transfected with constructs and an output (EYFP driven by a CRISPR activatable promoter). After 72 hours, fluorescence was measured by flow cytometry. Values are median or geometric mean+/−SD.

FIG. 14 demonstrates titration of HSV-1 helper virus. Titration was performed with virus collected from Vero 7b cells, grown to confluency. The Vero 7b cells were infected with serial dilutions of the virus for two hours and placed in a 1% methylcellulose media to prevent the spread of secondary infections. Localized plaques formed after about 4 days and cells were stained with crystal violet. If cells had been infected and subsequently detached, they were not stained by the crystal violet, leaving white circles on the plate, which were counted to get the titer of the virus. The well labels refer to the serial dilution of the virus, so the titer of this virus is approximately 6×10⁷ PFU/mL.

FIG. 15 presents phase and fluorescence images of HSV-1 EYFP infected and uninfected (control) organs of corti. Tissues were isolated a week before. Culture were infected 72 hours before with HSV-1 at the dosage of 10\hat{ }6 Pfu/organ of corti. Both images were taken with same exposure time.

FIG. 16 demonstrates staining of primary organs of corti isolated from mouse. Myosin VIIa is a marker for hair cells. 20× (top left) and 10× (top right). Sox2 (bottom left) is a marker for supporting cells. Phalloidin (bottom right), marks live cells. Hoescht stains nuclei. All images are 20× magnification (except for top right image which is 10×).

FIG. 17 demonstrates gene activation using gRNAs configured to activate Atoh1 and repress Hes1 and Hes5 in Neuro-2A and HEI-OC1 cell lines. gRNAs were assembled in an AAV backbone and tested in Neuro-2A cells. Graphs demonstrate a 16-fold increase in activation of Atoh1 expression using Atoh1 gRNA alone, 66-fold increase in activation of Atoh1 using Atoh1-Hes1 gRNA, and 84-fold increase in activation of Atoh1 expression using Atoh1-Hes5 gRNA.

FIG. 18 illustrates an exemplary method for delivering AAV-CRISPR and AAV-Cas9 constructs to the inner ear of neonatal mice. AAV-Atoh1-Hes1/Hes5 and AAV-Cas9 viruses were delivered to the inner ear of neonatal (P1-P3) mice via the posterior semicircular canal. Controls were injected with AAV-GFP or AAV-mCherry reporters. Auditory brainstem response (ABR) tests were first performed three weeks after injection at P21. A day later at P22, mice were administered with sisomicin and furosemide to induce hair cell damage. Second ABR tests were performed at P28, and a third ABR was performed at P60. After all rounds of ABR were completed, the mice were sacrificed and their cochleae were harvested in order to isolate organ of corti, stain them and analyze them.

FIG. 19 presents images of a side-by-side comparison of apical portions of organ of corti (OC) in an AAV-mCherry-injected ear and non-injected ear. Supporting cells are labeled with Sox2. Hair cells are labeled with MyoVIIa. Left: Many AAV transduced mCherry-positive cells. Hair cells labeled positive for both MyoVIIa and mCherry. Right: MyoVIIa positive hair cells are detected, but no mCherry positive AAV transduced cells.

FIG. 20 presents images of a side-by-side comparison of middle portions of an AAV-mCherry-injected ear and non-injected ear. Hair cells are labeled with MyoVIIa. The left middle portion shows many AAV-transduced mCherry-positive cells. Hair cells labeled positive for both MyoVIIa and mCherry. The right middle portion shows MyoVIIa positive hair cells but no mCherry positive AAV transduced cells.

FIG. 21 demonstrates inner hair cells (IHC) and Sox2+ supporting cells in apical sections of the inner ear of mice injected with AAV-Atoh1-Hes5-GFP and AAV-CRISPR-Cas9. Supporting cells are labeled with Sox2. Hair cells are labeled with MyoVIIa.

FIG. 22 demonstrates inner hair cells (IHC) and outer hair cells (OHC) in middle sections of the inner ear of mice injected with AAV-Atoh1-Hes5-GFP and AAV-CRISPR-Cas9. Supporting cells are labeled with Sox2. Hair cells are labeled with MyoVIIa.

FIG. 23 demonstrates inner hair cells (IHC) and outer hair cells (OHC) in middle sections of the inner ear of mice injected with AAV-Atoh1-Hes5-GFP and AAV-CRISPR-Cas9. Supporting cells are labeled with Sox2. Hair cells are labeled with MyoVIIa.

FIG. 24 demonstrates inner hair cells (IHC) and outer hair cells (OHC) in apical sections of the inner ear of AAV-mCherry injected mice.

FIG. 25 demonstrates assessment of functional improvement in mice injected with AAV-GFP or AAV-Atoh1-Hes1 and AAV-Cas9. Auditory brainstem response (ABR) tests were performed to assess hearing thresholds before and after antibiotic-induced hearing damage.

FIG. 26 demonstrates recovery of hearing in mice receiving AAV-Atoh1-Hes1 and AAV-Cas9, and in mice receiving AAV-Atoh1-Hes5 and AAV-Cas9. Four mice were injected with AAV-GFP. Another four mice were injected with AAV-Atoh1-Hes1 and AAV-Cas9. Another four mice were injected with AAV-Atoh1-Hes5 and AAV-Cas9. ABR results showed that the three groups had comparable hearing thresholds before antibiotic-induced hearing damage.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The systems and methods provided herein are based at least in part on the inventors' development of synthetic CRISPR-based regulatory circuits or systems useful for controllable and specific in vivo genome editing and transcriptional modulation in cells of the inner ear. Such regulatory systems or circuits, referred to herein as CRISPR hair cell classifiers. Embedded in the CRISPR hair cell classifiers are multiple layers of ribozyme-based kill switches that enable spatiotemporally-controlled transcriptional modulation and gene editing. In preferred embodiments, the CRISPR hair cell classifier is a single amplicon that can be packaged for in vivo delivery into a mammalian cell using a delivery vector such as an exosome, virus, or viral particle. As described herein, the multi-functionality of CRISPR hair cell classifiers makes the systems and methods described herein particularly advantageous for safely modulating endogenous gene expression with spatial and temporal control. In particular, the presence of embedded kill switches in the amplicon provides a means for targeting transcriptional modulation and/or gene editing in cells of the inner ear.

Synthetic CRISPR-based genetic circuits generally comprise multiple control elements (e.g., promoters, activators, repressor elements, insulators, silencers, response elements, introns, enhancers, transcriptional start sites, termination signals or poly(A) tails) that enable the genetic manipulation of cells for various purposes. For example, a biological circuit can be used to achieve the simple task of delivering a molecule to a cell that changes its differentiation state, inhibits or enhances a signaling pathway, or changes its growth rate. However, for more complex tasks such as engineering sophisticated control over cell function in cell-based therapies, controllable genetic circuits are useful. Aspects of the invention also encompass methods and uses of the compositions and systems described herein in genome engineering, e.g. for altering or manipulating the expression of one or more genes or the one or more gene products, in eukaryotic (e.g., mammalian) cells and prokaryotic cells in vitro, in vivo, or ex vivo.

Accordingly, in a first aspect, provided herein is a synthetic CRISPR hair cell classifier comprising a nucleotide sequence encoding a multifunctional Cas nuclease; at least two guide RNAs (gRNAs) comprising a first gRNA of 15 or less nucleotides (nt) in length (e.g., a 15-nt gRNA, a 14-nt gRNA, a 13-nt gRNA, a 12-nt gRNA, a 11-nt gRNA, a 10-nt gRNA, etc.) and a second gRNA of 16 or greater nt in length (e.g., a 16-nt gRNA, a 17-nt gRNA, a 18-nt gRNA, a 19-nt gRNA, a 20-nt gRNA, a 21-nt gRNA, etc.), wherein the first gRNA is complementary to at least a portion of an endogenous gene; and (c) a multilayered regulatory control element comprising (i) a first ligand-responsive ribozyme comprising a sensor component capable of detecting the presence or absence of a cell type-specific or small molecule signal and an actuator component; and (ii) a second layer comprising a ligand-responsive nuclease or regulatory polypeptide capable of cleaving and disabling the synthetic regulatory system in the presence of a second ligand; (ii) a second ligand-responsive ribozyme comprising a sensor component and an actuator component configured to promote expression of the second gRNA in the presence of a second ligand; wherein the nucleotide sequence encoding the Cas nuclease, the at least two gRNAs, and the multilayered regulatory control element comprise a single amplicon.

In particular, synthetic CRISPR hair cell classifiers described herein comprise multiple “kill switches” as a multilayered regulatory control element, where the control element “kill switches” enable safe regulation of gene expression by inactivating Cas nuclease activity under certain conditions and limit activation or repression of endogenous gene expression to cells of the inner ear (e.g., hair cells and supporting cells). Without being bound by any particular mechanism, theory, or mode of action, the synthetic CRISPR gene circuits described herein exploit the ablation of Cas nuclease activity (e.g., activity of nuclease Cas9) upon binding of the nuclease to a guide RNA (gRNA) having a 14-nt guide sequence rather than a 20-nt guide sequence.

Preferably, synthetic CRISPR-based hair cell classifiers provided herein are configured to modulate endogenous gene expression in a spatially and temporally controlled manner. Developed using logic-based design principles of synthetic biology and Cas/CRISPR system techniques, the synthetic CRISPR-based hair cell classifiers described herein limit in vivo expression of gRNAs to tissues or cells of interest, namely sensory epithelia of the inner ear (inner hair cells, outer hair cells, and inner ear supporting cells), and limit transcriptional modulation via the CRISPR-based system to particular time-points. For example, by embedding an inducible 20-nt gRNA in a synthetic CRISPR-based hair cell classifier comprising a 14-nt gRNA (for expression of the 20-nt gRNA under certain cell conditions), the circuit provides a platform for control over the timing of CRISPR functions. In such cases, the circuit employs multiple guide RNAs of different lengths a (e.g., 20-nt and 14-nt gRNAs) and is configured for simultaneous gene activation and repression (disruption) in a single cell.

In some cases, a synthetic CRISPR-based hair cell classifier as described herein is introduced into one or more cells of the inner ear. Sensory epithelia of the inner ear contain two major cell types: hair cells and supporting cells. There are two types of hair cells: inner hair cells and outer hair cells. Hair cells are innervated by neurons whose cell bodies sit outside the sensory epithelium, either in a sensory ganglion within the temporal bone (afferent neurons) or in the hindbrain (efferent neurons). Both hair cell types are found in the organ of Corti, which is located in the cochlea. The organ of Corti also comprises five different types of supporting cells organized in rows along the organ's length: Hensen's cells, Deiters' cells, pillar cells, inner phalangeal cells, and border cells. Differentiating hair cells prevent neighboring precursor cells from becoming hair cells through notch signaling; these precursors then assume a supporting cell fate. By disrupting this lateral inhibition mechanism by activating or repressing expression of various hair cell genes, it is possible to induce differentiation of supporting cells into hair cells.

In preferred embodiments, multiple cell type-specific ribozymes and external inducers (e.g., small molecule agents) are used to control where and when a therapeutic synthetic CRISPR-based hair cell classifier is functional. Ribozyme devices can function as ligand-responsive genetic switches or ribozyme switches, where ribozyme activity, and thus target RNA and protein levels, are modulated as a function of ligand concentration in the cell. For example, synthetic CRISPR hair cell classifiers of this disclosure can be spatially targeted to limit cleavage or transcriptional modulation to cells of interest (e.g., cells of the inner ear including inner ear sensory epithelia and neurons). In some cases, the first ligand responsive ribozyme comprises a TALE nuclease fused to a miRNA 183-responsive actuator, whereby, in the presence of miRNA 183, expression of the TALE nuclease is inhibited and the amplicon remains intact. Cells of the inner ear express low levels of miRNA 183. In some cases, the second ligand-responsive ribozyme comprises a TALE repressor fused to a small molecule-responsive degradation tag, whereby, in the presence of the small molecule, degradation of the TALE repressor promotes expression of the second gRNA and cleavage of the amplicon.

In some cases, a “kill switch” ribozyme comprises a SMASh degradation tag or another degradation/destabilization domain. In general, destabilizing domains are small protein domains that are unstable and degraded in the absence of ligand, but whose stability is rescued by binding to a high-affinity cell-permeable ligand. Genetic fusion of a destabilizing domain to a protein of interest results in degradation of the entire fusion. Addition of a ligand for the destabilizing domain protects the fusion from degradation and, in this manner, adds ligand-dependent stability to a protein of interest.

In some cases, it will be advantageous to deliver CRISPR-based hair cell classifiers at one time point and induce activity of the classifiers at a later time point. For example, CRISPR-based hair cell classifiers could be introduced into one or more cells of a neonate but not activated until a later time point (e.g., particular developmental stage). In such cases, it will be advantageous to configure a CRISPR-based hair cell classifier for temporal regulation such that introduction of an inducing agent (e.g., a chemical agent, light-induced agent) can reconstitute, for example, an aptamer-activator complex to modulate an endogenous gene of interest. By way of example, for chemical or light-based induction, aptamer MS2 can be fused with P65-HSF1 and a FKBP-rapamycin binding (FRB) domain or a mutant or variant thereof. In such cases, temporal control is maintained by the addition or removal of small molecule rapamycin (or a rapamycin analog or derivative) at specific time points. In certain embodiments, a ribozyme switch is responsive to a small molecule such as theophylline. In such cases, the presence of theophylline induces a conformational changes in the riboswitch in which the aptamer is bound to theophylline. The RBS is then released and able to promote protein translation.

In certain embodiments, provided herein is a synthetic regulatory system is a single amplicon comprising a multifunctional Cas nuclease, which is in some cases, fused to a functional domain, and at least two distinct gRNAs comprising a first gRNA of 15 or less nucleotides in length and a second gRNA of 16 or greater nucleotides in length, wherein the synthetic regulatory system modulates cleavage and/or transcription in a mammalian cell. As used herein, a “guide RNA” (gRNA) is nucleotide sequence that is complementary to at least a portion of a target gene. A gRNA target site also comprises a Protospacer Adjacent Motif (PAM) located immediately downstream from the target site. Examples of PAM sequence are known (see, e.g., Shah et al., RNA Biology 10 (5): 891-899, 2013). In some embodiments, the sequence of PAM is dependent upon the species of Cas nuclease used in the architecture.

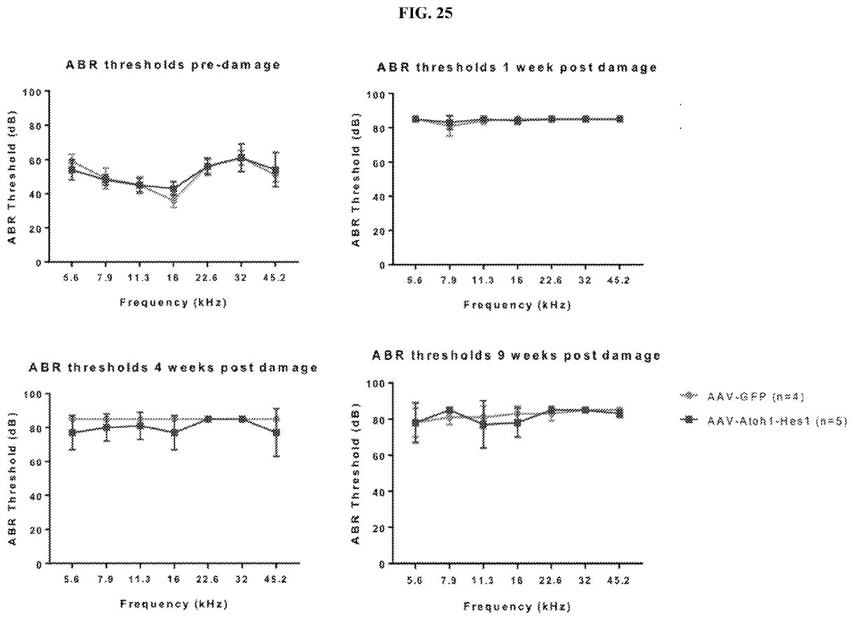

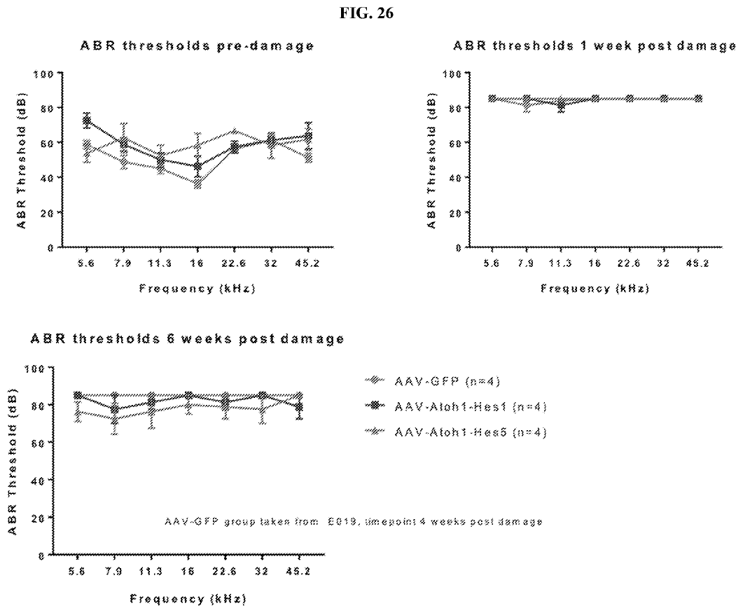

In certain embodiments, the first (truncated, 14 nt) gRNA is configured to target one or more endogenous genes including, without limitation, Atoh1 (Atonal basic helix-loop-helix (BHLH) Transcription Factor 1), BDNF (Brain-derived neurotrophic factor), HES1 (Hes Family BHLH Transcription Factor 1), HES5 (Hes Family BHLH Transcription Factor 5), and hepatocyte growth factor (HGF). Atoh1 and BDNF have been previously shown to be beneficial for hair cell regeneration and new nerve synapse formation. In addition, Atoh1 and BDNF loci can be targeted with minimal risk of off target binding to other sites of the genome. BDNF is a member of the neurotrophin family of growth factors, which are related to the canonical Nerve Growth Factor. Neurotrophic factors such as BDNF and HGF are found in the brain and in cells of the peripheral nervous system. For example, human HGF gene expression has been detected in the spiral ganglion cells (SGCs) of the inner ear. HES5 and HES5 encode members of a family of basic helix-loop-helix transcriptional repressors. In some cases, synthetic CRISPR hair cell classifiers of this disclosure are configured to activate expression of one or more of the above-described endogenous genes. In some cases, synthetic CRISPR hair cell classifiers of this disclosure are configured to repress expression of one or more endogenous genes such as HES1 and HES5. In some cases, a synthetic CRISPR hair cell classifier is configured to activate expression of Atoh1 and repress expression of HES1 and/or HES5. Referring to FIGS. 25 and 26, such activating and repressing hair cell circuits can be introduced into the inner ear of a mammal for at least partial restoration of hearing sensitivity following hearing loss.

It should be noted that the DNA-targeting sequence may or may not be 100% complementary to the target polynucleotide (e.g., gene) sequence. In certain embodiments, the DNA-targeting sequence is complementary to the target polynucleotide sequence over about 8-25 nucleotides (nts), about 12-22 nucleotides, about 14-20 nts, about 16-20 nts, about 18-20 nts, or about 8, 9, 10, 11, 12, 13, 14, 15, 16, 17, 18, 19, 20, 21, 22, 23, 24, or 25 nts. In certain embodiments, the complementary region comprises a continuous stretch of about 12-22 nts, preferably at the 3′ end of the DNA-targeting sequence. In certain embodiments, the 5′ end of the DNA-targeting sequence has up to 8 nucleotide mismatches with the target polynucleotide sequence. In certain embodiments, the DNA-binding sequence is about 50, 55, 60, 65, 70, 75, 80, 85, 90, 95, or 100% complementary to the target polynucleotide sequence.

To improve gene activation/repression effectiveness and scalability of the system, guide RNAs (gRNAs) are in some cases engineered to comprise a minimal hairpin aptamer. In some cases, the aptamer is appended to the tetraloop and stem loop of a gRNA. In some cases, the aptamer is capable of binding to the dimerized MS2 bacteriophage coat proteins. By fusing MS2 proteins with various activators such as p65 and HSF1 transactivation domains (e.g., MS2-p65-HSF1), a target-specific MS2-mediated gRNA can enhance recruitment of transcription factors to the target gene promoter and Cas complex.

In some cases, one or more gRNAs further comprise one or more RNA recognition motifs such as the MS2 binding motif, the COM binding motif, or the PP7 binding motif PP7 to which certain proteins (MS2 coat protein, COM, or PCP, respectively) bind. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. In some cases, the RNA recognition motif (e.g., MS2 binding motif, COM binding motif, or PP7 binding motif) is fused to an activation domain such as, for example, VPR or P65-HSF1. Other transcriptional activators include, without limitation, VP64. P65, HSF1, and MyoD1.

In some embodiments, one or more of the gRNAs is expression under the control of a RNA Pol II promoter or an RNA Pol II promoter. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. In some cases, synthetic promoters on the circuit are replaced with an endogenous gene promoter for control of endogenous gene expression. A promoter, generally, is a region of nucleic acid that initiates transcription of a nucleic acid encoding a product. A promoter may be located upstream (e.g., 0 bp to −100 bp, −30 bp, −75 bp, or −90 bp) from the transcriptional start site of a nucleic acid encoding a product, or a transcription start site may be located within a promoter. A promoter may have a length of 100-1000 nucleotide base pairs, or 50-2000 nucleotide base pairs. In some embodiments, promoters have a length of at least 2 kilobases (e.g., 2-5 kb, 2-4 kb, or 2-3 kb). In certain embodiments, gRNA expression from RNA pol II promoters can be modulated using Csy4 endoribonuclease-mediated cleavage. In some cases, multiple gRNAs are placed in tandem from a single coding region processed by Csy4.

In some cases, a CRISPR-based hair cell classifier is configured to comprise an endogenous gene promoter in place of a synthetic promoter. For example, a synthetic RNA Pol II or Pol III promoter can be swapped with a cell type- or context-specific promoter and interfaced with intracellular signaling, enabling multistep sensing and modulation of cellular behavior. In some cases, a transcriptional repression cascade comprises two, three, or four interconnected CRISPR transcriptional repression circuits (NAND logic gates).

In some embodiments, Cas nuclease is encoded from an engineered nucleic acid. For example, in certain embodiments, transcriptional modifiers are fused to Cas nuclease to enable site-specific transcriptional modifications. Various strategies can be used to engineer such fusion molecules. In some cases, transcriptional modulators are directly fused to the Cas nuclease protein. In other cases, the modulator is fused to another RNA binding protein such as MS2 bacteriophage coat protein in order to recruit the modulator to the Cas/gRNA/DNA complex.

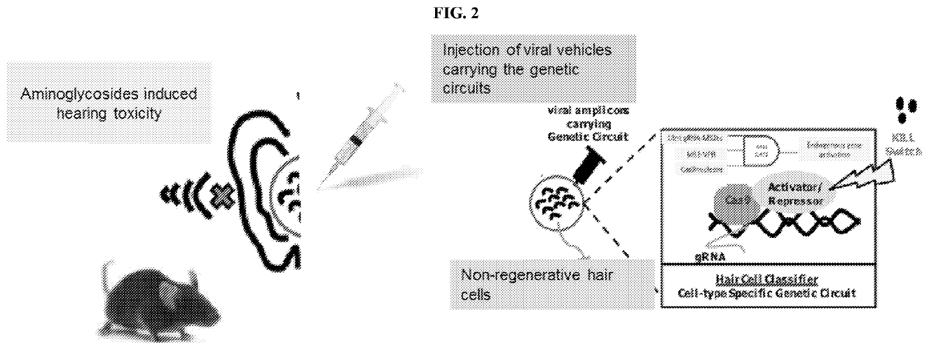

Referring to FIG. 2, an exemplary embodiment comprises a synthetic CRISPR-based classifier configured such that Cas9 nuclease and either MS2-VPR or MS2-P65-HSF1 are expressed from Pouf4f3 and Prestin promoters and bind 14-nt gRNAs having MS2 target sites. Multiple gRNAs are expressed from U6 promoters after further processing by a Csy4 endoribonuclease (from Myosin VIIa promoter). In this example, TALEN (Transcription Activator-Like Effector Nuclease) is expressed in cells that have low miRNA 183 cluster (outside inner ear) and destroys amplicons by cleaving the amplicon. In some cases, a TALE repressor (“TALER”) can be fused to a degradation tag (e.g., Small molecule-assisted shutoff (SMASh) domain or other degradation/destabilization domain) inhibits the expression of 20-nt gRNA. When a SMASh domain is used, the presence of a small molecule HCV protease inhibitor causes degradation of the repressor and, consequently, the expression of 20-nt gRNA and Cas9 nuclease-mediated destruction of amplicon. In other cases, the ligand or small molecule-responsive degradation system employs a Degron-Shield1 system. In such cases, the small molecule-responsive domain is a ligand-induced degradation domain (LID) comprising FK506-binding protein (FKBP) and a 19-amino-acid degron fused to the C terminus of FKBP. In the absence of the small molecule Shield-1, the degron is bound to the FKBP fusion protein and the protein is stable. When present, Shield-1 binds tightly to FKBP, displacing the degron and inducing rapid and processive degradation of the LID and the fused partner protein (e.g., Cas nuclease). See, for example, Bonger et al., Nat Chem Biol. 2011 August; 7(8): 531-537. Examples of suitable degrons have been well-characterized and tested in both cells and animals and include, but are not limited to, those degrons controlled by Shield-1, DHFR, auxins, and/or temperature. Non-limiting examples of suitable degrons are known in the art (e.g., Dohmen et al, Science, 263(5151): 1273-1276, 1994: “Heat-inducible degron: a method for constructing temperature-sensitive mutants”; Schoeber et al., Am. J. Physiol. Renal. Physiol., 296(1):F204-211, 2009: “Conditional fast expression and function of multimeric TRPV5 channels using Shield-1”; Chu et al., Bioorg. Med. Chem. Lett, 18(22): 5941-4, 2008: “Recent progress with FKBP-derived destabilizing domains”; Kanemaki, Pflugers Arch., 2012: “Frontiers of protein expression control with conditional degrons”; Yang et al., Mol. Cell, 48(4):487-8, 2012: “Titivated for destruction: the methyl degron”; Barbour et al, Biosci. Rep., 33(1), 2013: “Characterization of the bipartite degron that regulates ubiquitin-independent degradation of thymidylate synthase”; and Greussing et al, J. Vis. Exp., (69), 2012: “Monitoring of ubiquitin-proteasome activity in living cells using a Degron (dgn)-destabilized green fluorescent protein (GFP)-based reporter protein”; all of which are incorporated in their entirety by reference). In some cases, the Cas nuclease is fused to an activation or repression domain. In other cases, repression is achieved without the use of any repression domain but, rather, through Cas nuclease-mediated steric hindrance. The repression domain can comprise an aptamer sequence (e.g., MS2) fused to a repression domain such as, for example, a Kruppel associated box (KRAB) domain. Other repression domains include, without limitation, a methyl-CpG (mCpG) binding domain (e.g., binding domain for MeCP2) and KRAB-MeCP2.

In some cases, the Cas nuclease is Cas9 fused to a functional domain, where the nucleic acid sequence encoding Cas9 is split into two halves and fused to a FKBP/FRB domain. In other cases, the Cas nuclease is an allosteric Cas9, where the presence of a small molecule (e.g., ramapycin or tamoxifen) mediates assembly of functional Cas9 nuclease to enable temporal control over initiation of CRISPR function in vivo.

In some cases, the system comprises introducing into a single cell two CRISPR-based hair cell classifiers. The first cassette comprises a nucleotide sequence encoding a fusion polypeptide of a Cas9 nuclease and a 14-nt gRNA configured for gene modulation, where the fusion construct under the control of an inducible promoter. In some cases, the Cas9 nuclease is fused to a reporter polypeptide or polypeptide of interest. The reporter polypeptide may be a fluorescent polypeptide such as near infrared fluorescent protein (iRFP) (to monitor Cas9 protein dynamics). The second cassette comprises the “safety construct”—a 20-nt controllable gRNA. In some cases, an activator of the inducible promoter is provided by the safety construct. Activators can mediate or promote recruitment of the Pol II machinery to the CRISPR-Cas complex. The activator can be a zinc-finger protein fused to an activation domain such as a VP16 transcription activation domain or VP64 transcription activation domain. In certain embodiments, orthogonally acting protein-binding RNA aptamers such as MS2 are used for aptamer-mediated recruitment of an activator to the CRISPR-Cas complex. For example, an MS2-VPR fusion protein can be used to aid CRISPR-CAS/14-nt gRNA-mediated gene activation by means of aptamer-mediated recruitment of an activator to the CRISPR-Cas complex. In the presence of an inducer, the safety 20-nt gRNA is expressed, resulting in destruction of the 14-nt gRNA cassette.

In some cases, high ON/OFF ratios are achieved by using a modified U6-driven 14-nt gRNA cassette, where 20-nt gRNA target sites are inserted within both the U6 promoter site and body of the gRNA. This structure forms a second generation kill switch which enables full destruction of the 14-nt gRNA cassette upon expression of 20-nt gRNAs in vitro and in vivo. In some cases, such kill switches are expressed in vivo in, for example, a mouse liver, which is a frequent target in gene therapy and a tolerogenic immune environment.

The components of synthetic regulatory circuits described herein are preferably provided in a single amplicon. In some cases, however, the components may be in the form of two or more polynucleotide sequences. The synthetic regulatory circuit (e.g., CRISPR-based hair cell classifier) can be an engineered polynucleotide. As used herein, the terms “engineered nucleic acid” and “engineered polynucleotide” are used interchangeably and refer to a nucleic acid that has been designed and made using known in vitro techniques in the art. In some embodiments, an engineered polynucleotide, also referred to as a circuit herein, is a nucleic acid that is not isolated from the genome of an organism. In some embodiments, the engineered polynucleotide is introduced to a cell, plurality of cells, an organ or an organism to perform diverse functions (e.g., differentiation of cells, as sensors within cells, program a cell to act as a sensor, and delivery of selective cell-based therapies).

In some embodiments, the engineered polynucleotide comprises one or more promoters, such as an inducible promoter, constitutive promoter, or a tissue-specific or cell type-specific promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268: 1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only. Non-limiting examples of control elements include promoters, activators, repressor elements, insulators, silencers, response elements, introns, enhancers, transcriptional start sites, termination signals, linkers and poly(A) tails. Any combination of such control elements is contemplated herein (e.g., a promoter and an enhancer).

CRISPR systems belong to different classes, with different repeat patterns, sets of genes, and species ranges. A CRISPR enzyme is typically a type I or III CRISPR enzyme. The CRISPR system is derived advantageously from a type II CRISPR system. The type II CRISPR enzyme may be any Cas enzyme. The terms “Cas” and “CRISPR-associated Cas” are used interchangeably herein. The Cas enzyme can be any naturally-occurring nuclease as well as any chimeras, mutants, homologs, or orthologs. In some embodiments, one or more elements of a CRISPR system is derived from a particular organism comprising an endogenous CRISPR system, such as Streptococcus pyogenes (SP) CRISPR systems or Staphylococcus aureus (SA) CRISPR systems. The CRISPR system is a type II CRISPR system and the Cas enzyme is Cas9 or a catalytically inactive Cas9 (dCas9). Other non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. A comprehensive review of the Cas protein family is presented in Haft et al. (2005) Computational Biology, PLoS Comput. Biol. 1:e60. At least 41 CRISPR-associated (Cas) gene families have been described to date.

It will be understood that the CRISPR-Cas system as described herein is non-naturally occurring in a cell, i.e. engineered or exogenous to the cell. The CRISPR-Cas system as referred to herein has been introduced in a cell. Methods for introducing the CRISPR-Cas system in a cell are known in the art, and are further described herein elsewhere. The cell comprising the CRISPR-Cas system, or having the CRISPR-Cas system introduced, according to the invention comprises or is capable of expressing the individual components of the CRISPR-Cas system to establish a functional CRISPR complex, capable of modifying (such as cleaving) a target DNA sequence. Accordingly, as referred to herein, the cell comprising the CRISPR-Cas system can be a cell comprising the individual components of the CRISPR-Cas system to establish a functional CRISPR complex, capable of modifying (such as cleaving) a target DNA sequence. Alternatively, as referred to herein, and preferably, the cell comprising the CRISPR-Cas system can be a cell comprising one or more nucleic acid molecule encoding the individual components of the CRISPR-Cas system, which can be expressed in the cell to establish a functional CRISPR complex, capable of modifying (such as cleaving) a target DNA sequence.

In some embodiments, a synthetic CRISPR-based hair cell classifier as described herein may be introduced into a biological system (e.g., a virus, prokaryotic or eukaryotic cell, zygote, embryo, plant, or animal, e.g., non-human animal). A prokaryotic cell may be a bacterial cell. A eukaryotic cell may be, e.g., a fungal (e.g., yeast), invertebrate (e.g., insect, worm), plant, vertebrate (e.g., mammalian, avian) cell. A mammalian cell may be, e.g., a mouse, rat, non-human primate, or human cell. A cell may be of any type, tissue layer, tissue, or organ of origin. In some embodiments a cell may be, e.g., an immune system cell such as a lymphocyte or macrophage, a fibroblast, a muscle cell, a fat cell, an epithelial cell, or an endothelial cell. A cell may be a member of a cell line, which may be an immortalized mammalian cell line capable of proliferating indefinitely in culture.

In some cases, the CRISPR-based hair cell classifier is provided in a single amplicon that is packaged in a delivery vector for introduction into a cell (e.g., a mammalian cell). Any appropriate delivery vector can be used with the systems and methods described herein. For example, delivery vectors include exosomes, viruses (viral vectors), and viral particles. Preferably, the delivery vector is a viral vector, such as a lenti- or baculo- or preferably adeno-viral/adeno-associated viral vectors, but other means of delivery are known (such as yeast systems, microvesicles, gene guns/means of attaching vectors to gold nanoparticles) and are provided. In some embodiments, CRISPR-based hair cell classifier is delivered/introduced into a cell via liposomes, nanoparticles, exosomes, microvesicles, or a gene-gun. In certain preferred embodiments, CRISPR-based hair cell classifier or components thereof (e.g., gRNAs) are packaged for delivery to a cell in one or more viral delivery vectors. Suitable viral delivery vectors include, without limitation, adeno-viral/adeno-associated viral (AAV) vectors, lentiviral vectors, and Herpes Simplex Virus 1 (HSV-1) vectors. For example, a synthetic CRISPR-based hair cell classifier can be introduced into one or more Herpes simplex amplicon vectors.

Viral vectors and viral particles are commonly used viral delivery platforms for gene therapy. Therefore, for faster clinical translation, CRISPR-based hair cell classifiers as described herein are incorporated into Herpes simplex amplicon vectors. Preferably, a single carrier vector is used to achieve transfection of all synthetic genetic components to hair cells. Without being bound to any particular theory or mode of action, it is believed that the ability to control CRISPR functionality by the addition of ribozyme kill switches and gRNAs of various lengths as described herein is particularly advantageous due to the small DNA footprint of gRNA and limited pay load capacity of viral particles. In some cases, a Herpes simplex viral delivery system is used for delivery of engineered polynucleotides. In other cases, non-viral particles (e.g., exosomes) can be used for delivery. For example, controlled spatial transfection and/or destruction of CRISPR hair cell classifiers is achieved by assembling and packaging of all CRISPR-based hair cell classifier elements in HSV-1 amplicon vectors having large payload capacity (150 kb) and capable of infecting multiple inner ear cell types (e.g., nerve cells, hair cells, supporting cells). In some cases, HSV-1 infectivity can be first assayed in vitro using, as a model, murine organ of corti cultures and a fluorescent reporter molecule.

Given that DNA cleavage creates irreversible destruction of a gene, this “safety kill switch” mechanism can be effective to permanently shut down the CRISPR genetic circuit, should any adverse effect arise in vivo. However, size of CRISPR system and payload limitation of AAV vectors still necessitates co-administration of two AAV viruses in vivo. Accordingly, in another aspect, provided herein is a co-virus strategy, where the method comprises introducing into a single cell two AAVs. The first virus carries (i) a nucleotide sequence encoding a fusion polypeptide of a Cas9 nuclease, and (ii) a 14-nt gRNA configured for gene modulation. In some cases, the Cas9 nuclease is fused to a reporter polypeptide. The reporter polypeptide may be a fluorescent polypeptide such as near infrared fluorescent protein (iRFP) (to monitor Cas9 protein dynamics). The second virus is the “safety virus” and carries the 20-nt controllable gRNA. To ensure that expression of the Cas9-iRFP fusion polypeptide carried by the first virus occurs only in cells into which both AAVs are introduced, expression of the fusion polypeptide is under the control of an inducible promoter. In some cases, an activator of the inducible promoter is carried by the safety virus. Activators can mediate or promote recruitment of the Pol II machinery to the CRISPR-Cas complex. The activator can be a zinc-finger protein fused to an activation domain such as a VP16 transcription activation domain or VP64 transcription activation domain. In certain embodiments, orthogonally acting protein-binding RNA aptamers such as MS2 are used for aptamer-mediated recruitment of an activator to the CRISPR-Cas complex. For example, the second virus (safety virus) can carry an MS2-VPR fusion protein that aids in CRISPR-CAS and 14-nt gRNA-mediated gene activation by means of aptamer-mediated recruitment of an activator to the CRISPR-Cas complex. In the presence of an inducer, the safety 20-nt gRNA is expressed, resulting in destruction of the 14-nt gRNA cassette.

Applications of the CRISPR-based cell classifier circuits described herein include, without limitation, in vivo CRISPR-based precision gene therapies for treating chronic and acute conditions in a variety of cell types; a platform of CRISPR cell classifiers can be applied to other organs and expanded to other ear cell types, enabling reversal of many types of hearing loss; and in vivo interrogation of endogenous genes using CRISPR activators and repressors, including CRISPR-mediated endogenous gene activation. Other therapeutic applications include treating metabolic resilience in soldiers, improving survival from blood loss, chronic pain management, and enhancing human sensory performance.

Advantageous features of the systems and methods described herein include, without limitation, spatiotemporal limitations on CRISPR functionality via sensing of multiple cellular inputs; simultaneous activation of multiple endogenous genes using Cas9; transcriptional activation by a truncated gRNA (to inactivate Cas9 nuclease) and full-length gRNAs to induce Cas9 nuclease activity; multiple layers of genetic kill switches; use of a single carrier vector to improve transfection of all genetic components to hair cells; and the ability to spatially limit expression of gRNAs to tissues of interest.

In another aspect, provided herein is a polynucleotide comprising elements of a synthetic regulatory system described herein. In some cases, the polynucleotide is a single amplicon provided in a vector to, for example, a host cell. Host cells can be any type of cell of interest (e.g., a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage embryo). Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages, i.e., splittings, of the culture. Target cells can be unicellular organisms, or are grown in culture.

Methods of introducing a nucleic acid into a host cell are known in the art, and any known method can be used to introduce a nucleic acid (e.g., vector or expression construct) into a stem cell or progenitor cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al, Adv. Drug Deliv. Rev.), and the like.

So that the compositions, methods, and systems provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

As used herein, the terms “synthetic” and “engineered” are used interchangeably and refer to the aspect of having been manipulated by the hand of man.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent.

As used herein, “modifying” (“modify”) one or more target nucleic acid sequences refers to changing all or a portion of a (one or more) target nucleic acid sequence and includes the cleavage, introduction (insertion), replacement, and/or deletion (removal) of all or a portion of a target nucleic acid sequence. All or a portion of a target nucleic acid sequence can be completely or partially modified using the methods provided herein. For example, modifying a target nucleic acid sequence includes replacing all or a portion of a target nucleic acid sequence with one or more nucleotides (e.g., an exogenous nucleic acid sequence) or removing or deleting all or a portion (e.g., one or more nucleotides) of a target nucleic acid sequence. Modifying the one or more target nucleic acid sequences also includes introducing or inserting one or more nucleotides (e.g., an exogenous sequence) into (within) one or more target nucleic acid sequences.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1—Assessing In Vitro Functionality of CRISPR Activators for Activation of Endogenous Genes ATOH1 and BDNF

An HSV-1 based hair cell classifier was prepared in which Cas9, Csy4, and MS2 fused to an activation domain (VPR) are driven from cell type specific promoters to activate endogenous genes (ATOH1 and BDNF) using 14-nt gRNAs.

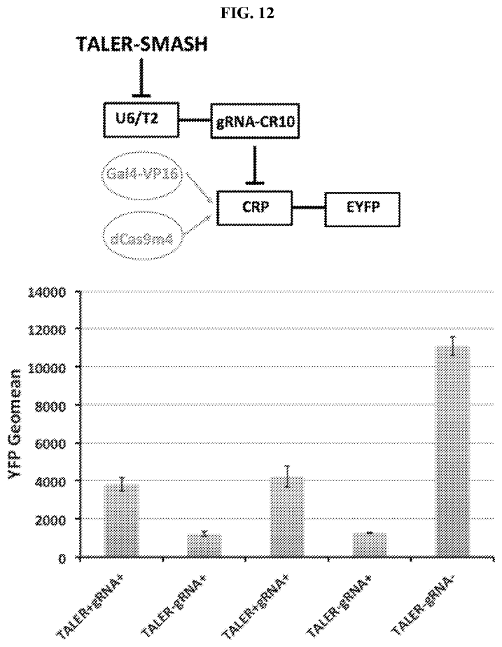

CRISPR activators for activation of endogenous gens (ATOH1) and BDNF were constructed and tested for functionality in vitro (which are necessary steps before moving to in vivo). We generated four truncated gRNAs that are designed to guide CRISPR activators to mouse BDNF and ATOH1 loci. To test the efficiency of these gRNAs for activation of these genes we tested the gRNAs in a mouse cell line that we had previously established (Raw cells). We transiently transfected these cells with our mixture of gRNA and Cas9 activators and measured ATOH1 and BDNF RNA level by qRT-PCR, 72 hours later. Raw cells were transfected with one, two or four gRNAs against ATOH1 or BSNF or three gRNAs against these genes (FIG. 12), together with the Cas9 activation complex. After 72 hours, cells were lysed for RNA extraction and qRT-PCR analysis. Our data demonstrated that a combination of 3 gRNAs per gene generate efficient gene activation for ATOH1 (FIG. 12). In particular, our data revealed that a triple gRNA combination generated more efficient activation, up to 24 fold over control for ATOH1 (FIG. 12c).

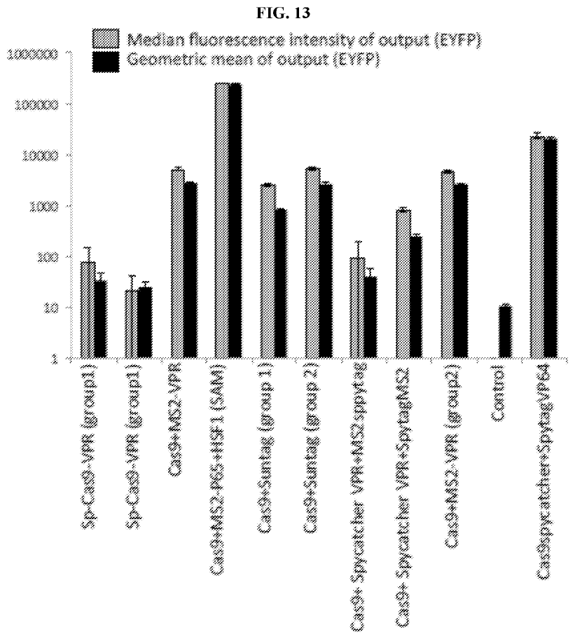

With regards to Cas9 activation module, we performed a side-by-side comparison of several Cas9 activators (Cas9 fusion with different activation domains) and measured the efficiency of these modules in activating the expression of a fluorescent output (FIG. 13). Side by side comparison of different CRISPR activators in HEK293 cells. Cells were transfected with constructs and an output (EYFP driven by a CRISPR activatable promoter). After 72 hours, fluorescence was measured using flow cytometry. We initial developed a tripartite strategy in which MS2 coat protein is fused to a dimerization domain (Spytag) and activation domain (VPR) fused to the other part of this peptide (VPR-spycatcher). See Proc Natl Acad Sci USA. 2012 109(12):E690-7. Spontaneous dimerization between spytag and spycatcher inside the cell was expected bring in the activation domain to Cas9 protein and the gRNA, which carries MS2 aptamer. However, after constructing and performing side by side comparison of this activator with other activators, we concluded that this strategy would not generate a strong activation module. We also concluded that a CRISPR/gRNA-directed Synergistic Activation Mediator (SAM) strategy in which two activation domains (P65 and HSF-1 catalytic domain) are directly fused to a MS2 coat protein would generate the most robust activation.

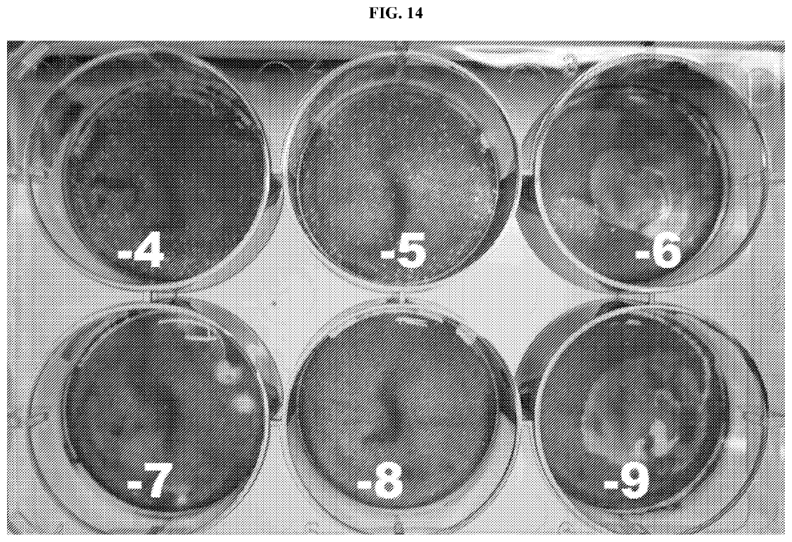

For HSV-1 amplicon technology, we performed a titration of HSV-1 helper virus. Titration was performed with collected virus. Vero 7b cells, grown to confluency were infected with serial dilutions of the virus for two hours and placed in a 1% methylcellulose media to prevent the spread of secondary infections. Localized plaques formed after about 4 days and cells were stained with crystal violet. If cells had been infected but subsequently detached, they were not stained by the crystal violet, leaving white circles on the plate, which were counted to get the titer of the virus. Referring to FIG. 14, the well labels refer to the serial dilution of the virus. Titer of HSV-1 was approximately 6×10⁷ PFU/mL.

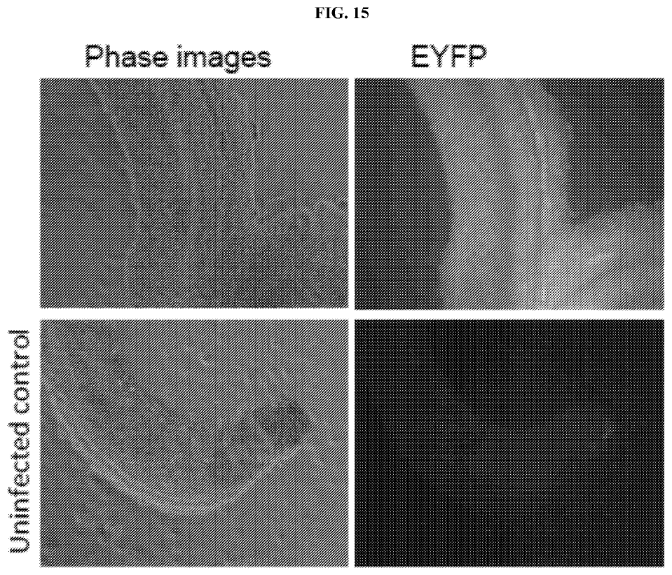

We obtained premade HSV-1 viruses carrying yellow fluorescent protein (EYFP). Subsequently, we isolated organ of corti from neonate mouse (P5) and infected the cultures with HSV-1 viruses with the dosage of 10\hat{ }4 and 10\hat{ }6 pfu/organ of corti. For phase and fluorescence images of HSV-1 EYFP infected and uninfected control organs of corti. Tissues were isolated a week before. Cultures were infected 72 hours before with HSV-1 at the dosage of 10\hat{ }6 Pfu/organ of corti. Our data suggest that it is possible to infect organ of corti with a HSV-1 amplicon (FIG. 15).

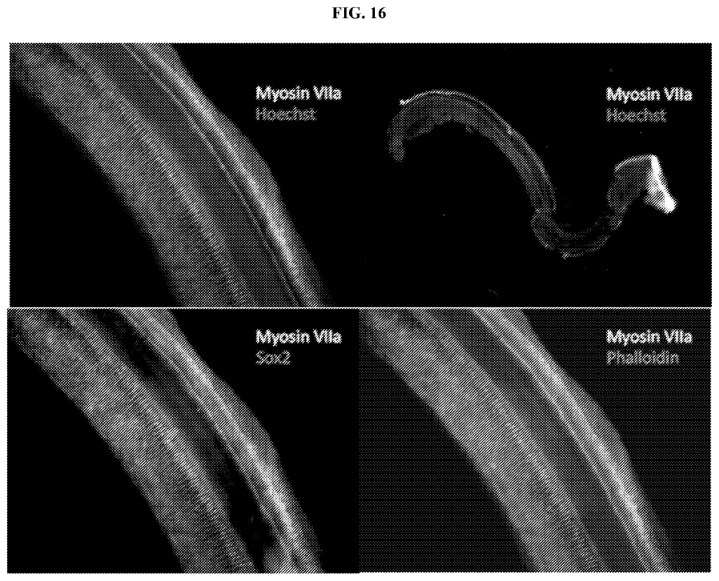

An in vitro model of mouse primary organs of corti will be established in HEI-OC1 cells (House Ear Institute-Organ of Corti 1 cells), which are one of the few mouse auditory cell lines that are currently used in auditory research. We have been successful to maintain the organ of cortis alive in culture for 8-10 days and have established the staining protocols for evaluation of organ of corti (FIG. 16).

SAM versus MS2-VPR system for Cas9 activation: Our data indicates that best activation can be achieved through binding of MS2 coat protein with P65 and HSF-1 activation domains and we think that this strategy (SAM method) is the best option to achieve functionality in vitro. This strategy is compatible with a dual promoter (cell type specific promoter) strategy in which one promoter drives Cas9 nuclease and another drives MS2-P65-HSF1. In case, triple promoter is required, we can employ split Cas9 nucleases and drive each half from one cell type specific promoter. We believe that a dual promoter can give sufficient spatial limitation while maintaining the high level of efficiency for endogenous activation.

### Example 2—CRISPR Based In Vivo Activation of Atoh1 and Repression of Hes1 and Hes5 to Treat Hearing Loss

Gene therapy based treatment of hearing loss focuses on strategies to reprogram supporting cells to hair cells in inner ear. The reprogramming relies on downregulation of Notch signaling pathway by manipulating its components. Atoh1 is the master regulator of hair cell differentiation in inner ear while Hes1 and Hes5 undermine the process. This example describes simultaneous epigenetic and genetic manipulation to downregulate the Notch signaling pathway. More specifically, this section demonstrates use of AAV viruses having 14 nt gRNA with a SAM site for Atoh1 activation and another 14 nt gRNA, which leads to steric blocking, for Hes1 or Hes5 repression.

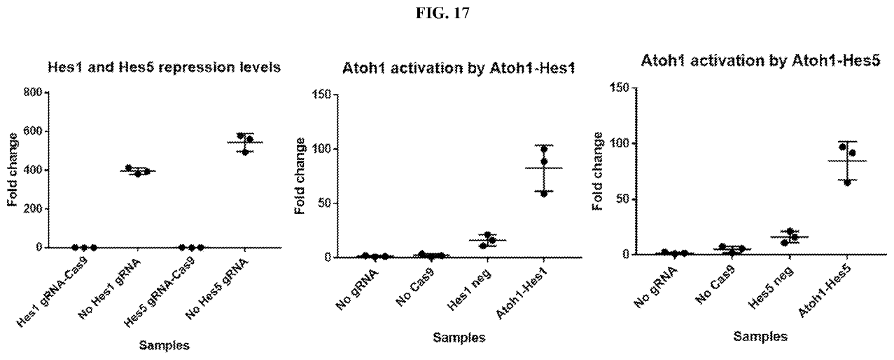

First, gRNAs were screened for those that activate Atoh1 and repress Hes1 and Hes5 in multiple cell lines including Neuro-2A and HEI-OC1. These gRNAs were then assembled in AAV backbone and tested again in a Neuro-2A cell line. As shown in FIG. 17, we observed a 16-fold activation of Atoh1 expression with an Atoh1 gRNA alone, a 66-fold activation with Atoh1-Hes1, and a 84-fold activation with an Atoh1-Hes5 gRNA construct.

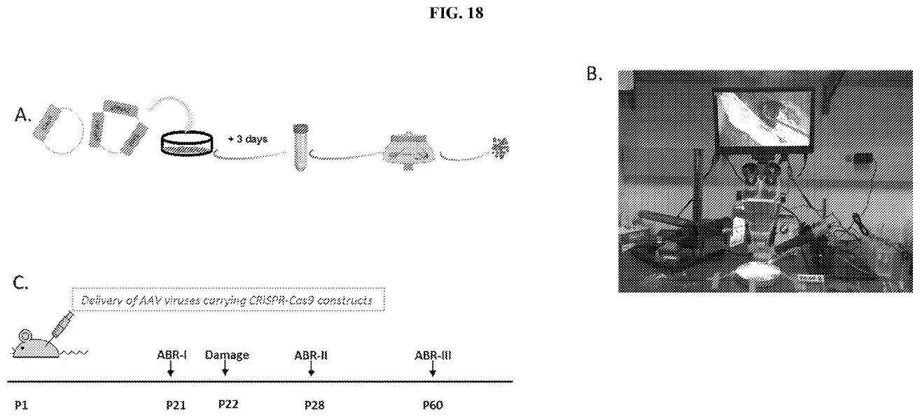

FIGS. 18A-18C illustrate exemplary methods of producing AAV (A), microinjecting the AAV constructs (B), and delivering AAV-CRISPR and AAV-Cas9 constructs to the inner ear of neonatal mice (C). Auditory brainstem response (ABR) tests, which measure auditory nerve reactions in response to sounds, were performed prior to inducing hearing damage with sisomicin and furosemide, and at second and third time-points following hearing damage.

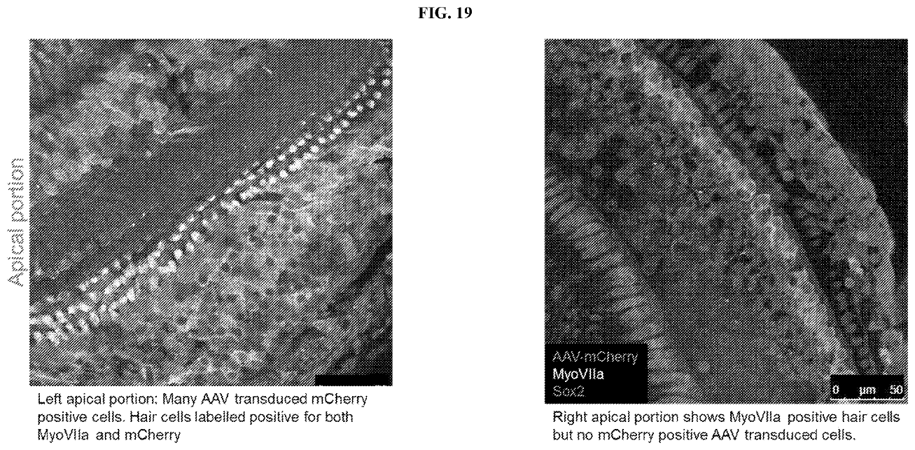

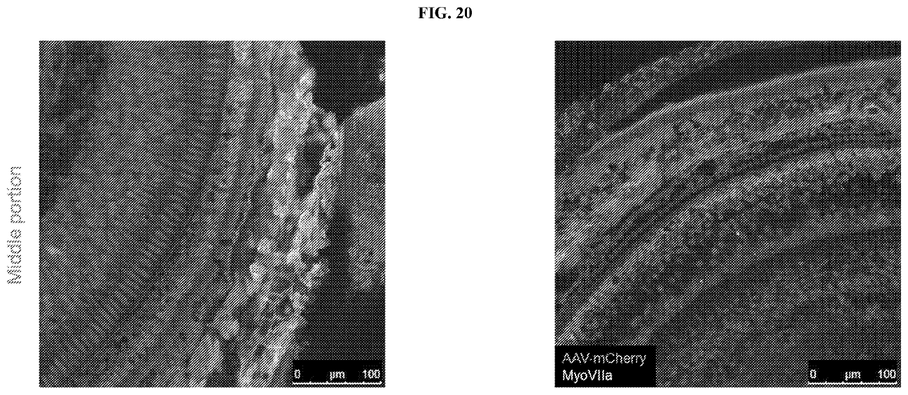

Side-by-side comparisons of apical portions of the organ of corti from AAV-mCherry-injected (left) and non-injected ears (right) revealed loss of hair cells in the outer rows in both ears while the inner row was intact in each (FIG. 19). In particular, we observed many AAV transduced mCherry positive cells, and hair cells were positive for both MyoVIIa and mCherry in the injected ear. In the non-injected ear (right), MyoVIIa+ hair cells were observed, but no mCherry+ cells were observed. A similar loss of hair cells in the outer rows was observed in side-by-side comparisons of middle portions (FIG. 20).

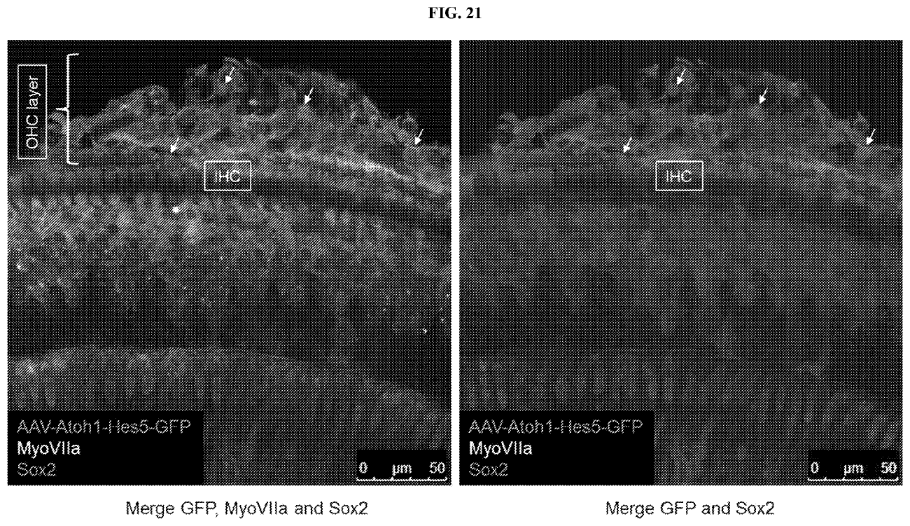

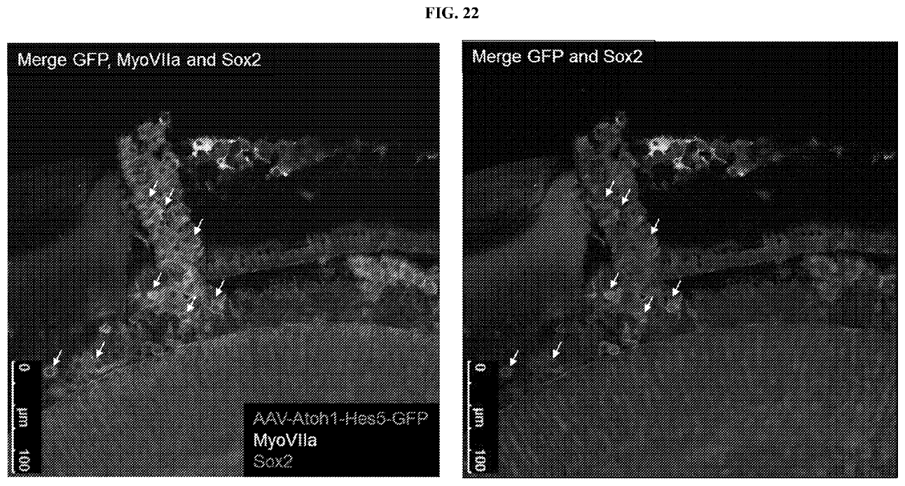

FIG. 21 demonstrates apical portions of inner ear following injection of AAV-CRISPR-Cas9 and AAV-Atoh1-Hes5. Some inner ear hair cells were transduced with AAV. Some of the transduced supporting cells were positive for MyoVIIa (hair cell marker) as well (indicated by arrowheads). As shown in FIG. 22, middle portions contained some transduced supporting cells that were positive for hair cell marker MyoVIIa (indicated by arrowheads).

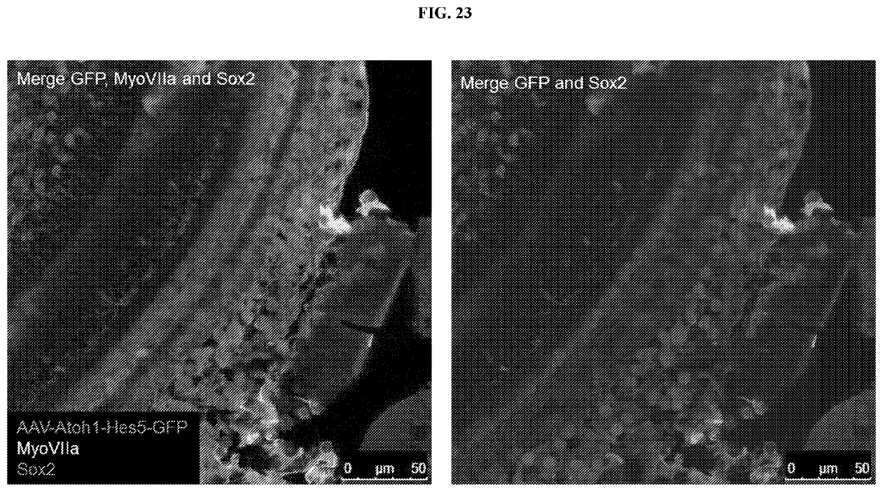

FIG. 23 demonstrates transduced inner hair cells (IHC) and outer hair cells (OHC). Of 40 inner hair cells, 20 were transduced. Of 30 outer hair cells in layer 1, 20 were transduced. Of 25 outer hair cells in the 2nd layer, 15 were transduced. Of 17 outer hair cells of the third layer, 8 were transduced. OHC layers 2 and 3 exhibit strong staining for Sox2, a marker of supporting cells (SCs). It is possible that OHCs in layers 2 and 3 were completely damaged and now SCs are taking their place. The round morphology of the Sox2+ cells is different from the elongated morphology of OHCs of layer 1. They also appear to be in a slightly different focal plane. It is possible that these cells are in transition, converting from SCs to HCs, but are not yet terminally differentiated.

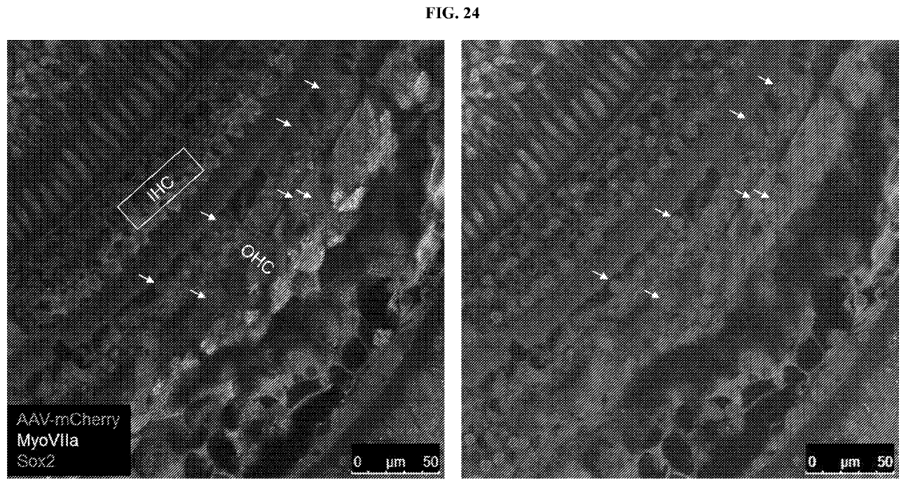

In AAV-mCherry controls (FIG. 24), all cells in OHC region are lost, presumably due antibiotic-induced damage. Some supporting cells in that region were transduced with AAV-mCherry, but those cells do not stain positive for hair cell marker MyoVIIa (indicated by arrows). Of 22 inner hair cells, 20 were transduced. Only 4 transduced cells were observed in the outer hair cell layers. 14 supporting cells (Sox2+) were observed in the OHC region.

ABR tests were performed to assess functional improvement. For these assays, four mice were injected with AAV-GFP, and five were injected with AAV-Atoh1-Hes1 and AAV-Cas9. ABR results (FIG. 25) showed that the two groups have similar hearing thresholds before damage. One week post damage, both groups appeared to be least sensitive to hearing. Four weeks post damage, we observed some recovery of hearing in mice of the AAV-Atoh1-Hes1+AAV-Cas9 group, while no hearing recovery was observed in mice of the AAV-GFP group. At nine weeks post damage, mice receiving AAV-Atoh1-Hes1+AAV-Cas9 retained the recovery of hearing thresholds at a few frequencies.

In another experiment, 4 mice were injected with AAV-GFP, 4 were injected with AAV-Atoh1-Hes1 and AAV-Cas9, and 4 were injected with AAV-Atoh1-Hes5 and AAV-Cas9. ABR results (FIG. 26) showed that the three groups had comparable hearing thresholds before antibiotic induced-hearing damage. One week post damage, all three groups appeared to be least sensitive to hearing. Six weeks post damage, we observed some recovery of hearing in AAV-Atoh1-Hes1 and AAV-Cas9 group and AAV-Atoh1-Hes5 and AAV-Cas9 group, while no recovery was observed in mice of the AAV-GFP group.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

We claim:

1. A synthetic regulatory system comprising
(a) a nucleotide sequence encoding a Cas nuclease;
(b) at least two guide RNAs (gRNAs) comprising a first gRNA of 15 or less nucleotides (nt) in length and a second gRNA of 16 or greater nt in length, wherein the first gRNA is complementary to at least a portion of an endogenous gene, wherein the first gRNA is operably linked to a CRISPR-responsive promoter; and
(c) a multilayered regulatory control element comprising (i) a first ligand-responsive ribozyme comprising a sensor component capable of detecting the presence or absence of a cell type-specific or small molecule signal and an actuator component; and (ii) a second layer comprising a ligand-responsive nuclease or regulatory polypeptide capable of cleaving and disabling the synthetic regulatory system in the presence of a second ligand;
wherein the nucleotide sequence encoding the Cas nuclease, the at least two gRNAs, and the multilayered regulatory control element comprise a single amplicon.

2. The system of claim 1, wherein the second layer comprises a TALE nuclease or a zinc finger nuclease (ZFN) fused to a miRNA 183-responsive actuator, whereby, in the presence of miRNA 183, expression of the TALE nuclease or ZFN is inhibited and the amplicon remains intact.

3. The system of claim 1, wherein the second layer comprises a TALE or Tet repressor fused to a small molecule-responsive degradation tag, whereby, in the presence of the small molecule, degradation of the TALE or Tet repressor promotes expression of the first gRNA and cleavage of the amplicon.

4. The system of claim 3, wherein the small molecule-responsive degradation tag is a SMASH tag or Degron-Shield1 system.

5. The system of claim 1, wherein the Cas nuclease is Cas9.

6. The system of claim 1, wherein the endogenous gene is selected from the group consisting of Atoh, BDNF, Hes1, Hes5, and HGF.

7. The system of claim 1, wherein the amplicon further comprises a nucleotide sequence encoding a MS2 bacteriophage coat protein and the first gRNA comprises a MS2 target sequence.

8. The system of claim 7, wherein the MS2 bacteriophage coat protein is fused to transcriptional activation domain VPR-P65-HSF1.

9. The system of claim 1, wherein one or more of the at least two gRNAs are operably linked to a U6 promoter.

10. The system of claim 1, wherein the Cas nuclease is fused to a functional domain selected from the group consisting of a transcriptional activator, a transcriptional repressor, methyltransferase and a nuclease cleavage domain.

11. The system of claim 10, wherein the Cas nuclease is Cas9 fused to a functional domain, wherein the nucleic acid sequence encoding Cas9 is split into two halves and fused to a FKBP/FRB domain

12. The system of claim 10, wherein the Cas nuclease is an allosteric Cas9, wherein the presence of ramapycin or tamoxifen mediates assembly of functional Cas9 nuclease to enable temporal control over initiation of CRISPR function in vivo.

13. The system of claim 10, wherein the functional domain comprises one or more transcriptional activators selected from the group consisting of VPR, VP64, P65, and HSF1.

14. The system of claim 10, wherein the functional domain comprises one or more transcriptional repressors selected from the group consisting of Kruppel associated box (KRAB) and KRAB-MeCP2.

15. The system of claim 1, further comprising a delivery vector.

16. The system of claim 15, wherein the delivery vector is an exosome.

17. The system of claim 16, wherein the exosome comprises a cell-specific or tissue-specific ligand or receptor.

18. The system of claim 15, wherein the delivery vector is a viral delivery vector selected from the group consisting of Herpes Simplex virus, retrovirus, lentivirus, adenovirus, adeno-associated virus, and baculovirus DNA.

19. The system of claim 18, wherein the viral delivery vector is Herpes Simplex Virus 1 (HSV1).

20. The system of claim 1, wherein the Cas nuclease is a S. aureus Cas9 nuclease or a S. pyogenes Cas9 nuclease.

21. A method of modulating endogenous gene expression in an inner ear cell, the method comprising introducing into an inner ear cell the synthetic regulatory system of claim 1, wherein the single amplicon is provided in a delivery vector.

22. The method of claim 21, wherein the inner ear cell is an inner hair cell, an outer hair cell, or a inner ear supporting cell.

23. The method of claim 21, wherein modulating comprises one or more of gene activation, gene repression, and gene inactivation.

24. The method of claim 21, wherein the delivery vector is an exosome.

25. The method of claim 24, wherein the exosome comprises a cell-specific or tissue-specific ligand or receptor.

26. The method of claim 21, wherein the delivery vector is a viral delivery vector selected from the group consisting of Herpes Simplex virus, retrovirus, lentivirus, adenovirus, and adeno-associated virus.

27. The method of claim 26, wherein the viral delivery vector is Herpes Simplex Virus 1 (HSV1).

28. The method of claim 21, wherein introducing comprises transfection or electroporation.

29. A polynucleotide sequence comprising
(a) a nucleotide sequence encoding a Cas nuclease;
(b) at least two guide RNAs (gRNAs) comprising a first gRNA of 15 or less nucleotides (nt) in length and a second gRNA of 16 or greater nt in length, wherein the first gRNA is complementary to at least a portion of an endogenous gene, wherein the first gRNA is operably linked to a CRISPR-responsive promoter; and
(c) a multilayered regulatory control element comprising (i) a first ligand-responsive ribozyme comprising a sensor component capable of detecting the presence or absence of a cell type-specific or small molecule signal and an actuator component; and (ii) a second layer comprising a ligand-responsive nuclease or regulatory polypeptide capable of cleaving and disabling the synthetic regulatory system in the presence of a second ligand;
wherein the nucleotide sequence encoding the Cas nuclease, the at least two gRNAs, and the multilayered regulatory control element comprise a single amplicon.

30. A vector comprising the polynucleotide sequence of claim 29.

31. A host cell comprising the vector of claim 30.
